(19) United States
(12) Patent Application Publication
(10) Pub. No.: US 2004/0215278 A1
(43) Pub. Date: Oct. 28, 2004

(54) Method and apparatus for locally upgrading implanted reprogrammable medical devices

(76) Inventors: Wim Stegink, Laren (NL); Henk Westendorp, Zutphen (NL); Paul Visser, Doesburg (NL); Xander Evers, Dieren (NL); Peter Van Geffen, Apeldoorn (NL); Gertwin Anbergen, Oss (NL)

Correspondence Address: MEDTRONIC, INC., 710 MEDTRONIC PARKWAY NE, MS-LC340, MINNEAPOLIS, MN, 55432-5604, US

(21) Appl. No.: 10/424,389

(22) Filed: Apr. 25, 2003

(51) Int. Cl. A61N 1/362; A61N 1/08

(52) U.S. Cl. 607/30; 607/60

(57) ABSTRACT

A method for enabling a programming device to upload a distributed software upgrade into a reprogrammable device having a unique serial number, the software upgrade having a unique upgrade identifier. The method comprises the steps of calculating a first enabling code as a function of the unique upgrade identifier and the unique serial number and comparing the first enabling code to a second enabling code received from a registry. Also disclosed is an apparatus for changing a heart-stimulating waveform comprising an implanted reprogrammable medical device (IRMD) having a unique serial number, an IRMD software upgrade program having a unique media identifier, and a programming device for uploading the IRMD software upgrade program into the IRMD.

### FIELD OF THE INVENTION

This invention relates generally to a method and apparatus for locally upgrading reprogrammable devices, and more specifically to upgrading implanted medical devices such as pacemakers.

### BACKGROUND OF THE INVENTION

Previously, methods and apparatuses have been developed for implanting programmable devices. U.S. Pat. No. 5,360,437 issued Nov. 1, 1994 discloses a pacemaker that can be sent new instructions without invasive procedures. U.S. Pat. No. 5,725,559 issued Mar. 10, 1998 discloses a pacemaker that stores a plurality of programs, any one of which may be selected through non-invasive means. In that case, the programs are loaded at the time the device is implanted.

More recently, methods and apparatus have been developed for upgrading implanted reprogrammable devices. One example is shown and described in U.S. Pat. No. 6,363,282 B1 issued Mar. 26, 2002 which describes downloading upgrade software over the internet or wirelessly from a remote software depository. Unfortunately, the use of the internet raises certain concerns about the overall reliability of systems which depend thereon, especially systems upon which an individual's health and welfare may depend.

### BRIEF SUMMARY OF THE INVENTION

According to a broad aspect of the invention there is provided a method for enabling a programming device to upload a distributed software upgrade into a reprogrammable device having a unique serial number, the software upgrade having a unique upgrade identifier. The method comprises the steps of calculating a first enabling code as a function of the unique upgrade identifier and the unique serial number and comparing the first enabling code to a second enabling code received from a registry. Also disclosed is an apparatus for changing a heart-stimulating waveform comprising an implanted reprogrammable medical device (IRMD) having a unique serial number, an IRMD software upgrade program having a unique media identifier, and a programming device for uploading the IRMD software upgrade program into the IRMD.

### BRIEF DESCRIPTION OF THE DRAWINGS

FIG. 1 illustrates an exemplary apparatus for locally upgrading implanted reprogrammable devices; and

FIG. 2 is a block diagram illustrating an exemplary method for locally upgrading implanted reprogrammable devices.

### DETAILED DESCRIPTION OF THE INVENTION

The following detailed description is merely exemplary in nature and is not intended to limit the invention or the application and uses of the invention. Furthermore, there is no intention to be bound by any theory presented in the preceding background of the invention or the following detailed description.

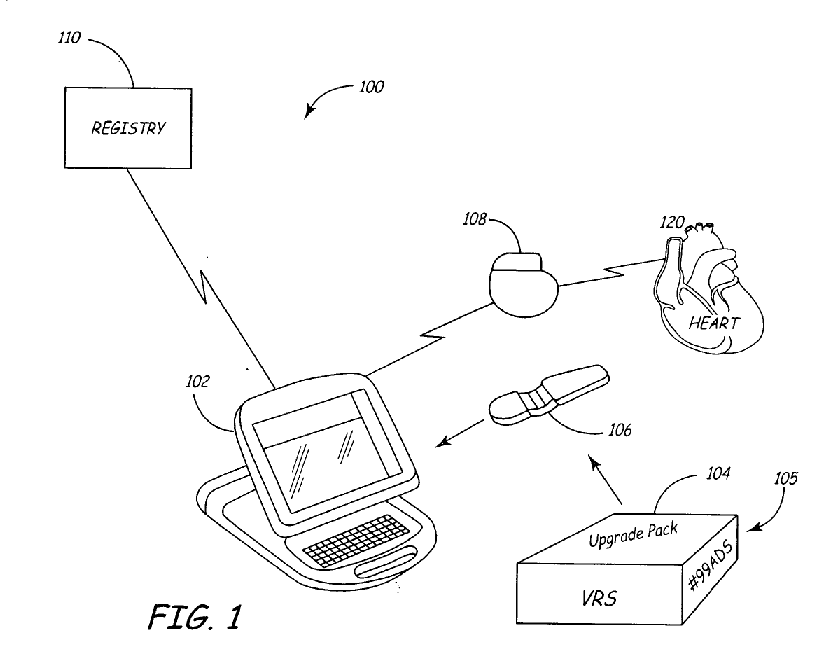

FIG. 1 illustrates an exemplary apparatus 100 for upgrading a reprogrammable device 108 such as implanted reprogrammable medical device (IRMD) (e.g., a pacemaker) 108 linked to a patient's heart 120. Apparatus 100 includes a programming device 102 linked to a registry 110, to IRMD 108, and to machine-readable media 106 containing a software upgrade for IRMD 108. Programming device 102 is configured to read media 106 and, given a correct enabling code, to upload the software upgrade from media 106 to IRMD 108.

Media 106 is part of upgrade pack 104, which may further include a manual of instructions. Media 106 has a unique media identifier 105 displayed on the upgrade pack 104 and/or the media 106, or other contents of the upgrade pack. Media 106 may be a memory stick, as shown, a floppy disk, a CD-ROM, or similar machine readable media.

The link between the programming device 102 and registry 110 enables programming device 102 to transmit copies of the IRMD serial number and the unique media identifier 105 to the registry 110 and to receive an enabling code from registry 110. The enabling code is a “marriage” code, in that it is a function of both the IRMD serial number and the unique media identifier 105. The link between the programming device 102 and the registry may be an internet, intranet, telephonic, or similar data link.

The link between the programming device 102 and the IRMD 108 enables downloading data from the IRMD, including the IRMD's unique serial number and data used for medical diagnosis. Some data analysis steps supporting medical diagnosis may be retrieved by software in the programming device upon receipt of the downloaded data from IRMD 108. The programming device 102 may be a dedicated instrument or a specially configured personal computer. Programming device 102 is operable to upload software upgrades to the IRMD 108, download data from IRMD 108, link to a registry 110, and receive inputs through a user interface such as a keyboard. The programming device 102 may itself be programmed with new software.

The IRMD 108 generates waveforms for stimulating heart 120 in various ways. The therapeutic waveforms, including their shape, frequency, amplitude, triggers, and timing, are determined by software within the IRMD 108. It is this waveform generating software that is the subject of the upgrade. The IRMD 108 may have one or more sensors for sensing heart problems, such as atrial fibrillation, which may be used as triggers for waveform generation from the IRMD 108. IRMD 108 also records data regarding heart and IRMD activities. This recorded data may be non-invasively downloaded.

Registry 110 is a vendor-controlled facility operable to calculate an enabling code from an IRMD serial number and a unique media identifier 105 using the same function as the programming device 102. The registry 110 may be a person with a calculator and a telephone, an automated web site, or a computer having software responsive to calculate enabling codes. The registry 110 may also record associations between upgrade software and IRMDs 108, and data related to the association, such as a date.

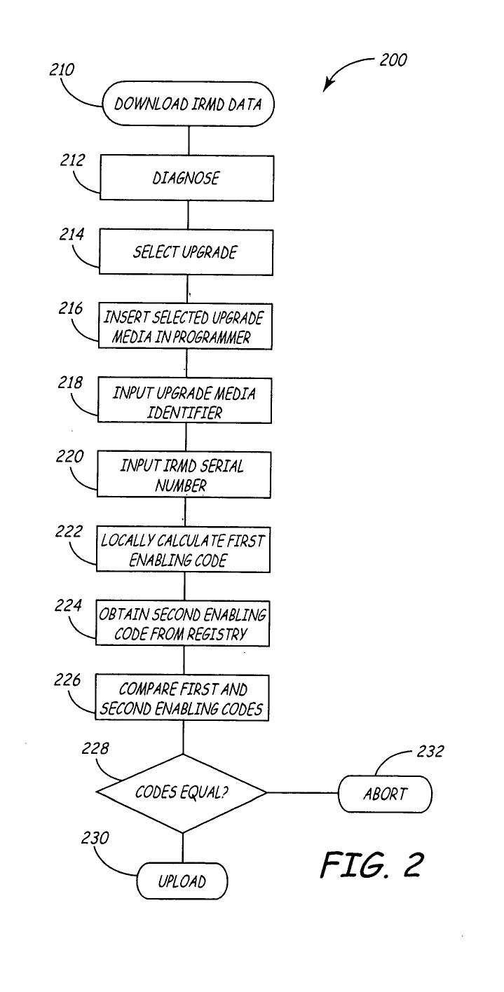

FIG. 2 shows exemplary process 200 for uploading a software upgrade to an IRMD 108. In the first step 210, data from the IRMD is downloaded into the programming device 102. In step 212, data analysis and medical expertise of the user combine to diagnose any conditions responsive to a new therapy. If a new therapy is called for by the diagnosis, the appropriate upgrade is selected by the user as is shown at 214. The user (typically a doctor) unwraps upgrade package 104, removes the memory stick 106, and inserts the memory stick 106 in programming device 102 as is shown at 216. Installation wizard software on the memory stick may automatically load and present screen prompts for inputs from the user.

As is shown at 218, the user inputs the media identifier 105 by typing the media identifier 105 as an alphanumeric text string into a window associated with a screen prompt. The user then enters the IRMD serial number in a similar fashion as is shown at 220. In an alternate embodiment, the installation wizard loads the downloaded serial number into an editable window and requests verification by the user. The media identifier 105 is contained in the media 106, and is read and pre-loaded into another input window by the installation wizard. Steps 218 and 220 may be done in any order.

In step 222, the programming device 102 calculates a first enabling code from the media identifier 105 and the serial number of IRMD 108. The programming device 102 does not display the first enabling code to the user. Rather, the user is prompted to obtain and enter an enabling code from registry 110. In step 224, the user contacts registry 110, provides the IRMD serial number and the unique media identifier and receives a second enabling code calculated at the registry 110. The user may enter the second enabling code in a window associated with the prompt, or the installation wizard may pre-load the entry window for review if the second enabling code is received electronically. The first and second enabling codes are compared in step 226, and the upload is enabled (230) or aborted (232) depending on the outcome of the comparison. If the first and second enabling codes compare, the upload takes place. If not, the upload is aborted. In a first embodiment, the aborted upload may not be retried, and another upgrade package 104 may have to be opened. In an alternate embodiment, a predetermined number of attempts may be possible.

Any number of functions may be used to calculate an enabling code. In certain embodiments, different functions may be selected based, for example, on different prefixes to media identifiers. In other embodiments, the function and its inputs may have a relationship ensuring that only a compatible software upgrade will be enabled. For example, a new line of IRMDs may have upgrades that are not backwardly compatible to older IRMDs, and those upgrades may have serial numbers that produce proscribed numbers an enabling code is calculated for a newer upgrade and an older IRMD. Those of ordinary skill in the art may appreciate that a variety of functions may be used.

Thus, embodiments of the method and apparatus for locally upgrading implanted reprogrammable devices are disclosed. One skilled in the art will appreciate that the present invention can be practiced with embodiments other than those disclosed. The disclosed embodiments are presented for purposes of illustration and not limitation, and the present invention is limited only by the claims that follow.

We claim:

1. A method for uploading a distributed software upgrade to a reprogrammable medical device having a serial number from a programming device linked to a registry, said software upgrade having a upgrade identifier, the method comprising the steps of:
calculating a first enabling code as a function of said upgrade identifier and said serial number; and
comparing said first enabling code to a second enabling code received from the registry.

2. The method of claim 1, wherein the step of comparing comprises the step of receiving said second enabling code at said programming device.

3. The method of claim 2, wherein the step of receiving said second enabling code further comprises linking said programming device to said registry.

4. The method of claim 2, further comprising the step of receiving said unique upgrade identifier and said unique serial number into said programming device.

5. The method of claim 1, further comprising the step of uploading said upgrade software into said reprogrammable device if said first enabling code matches said second enabling code.

6. The method of claim 1, further comprising creating a relationship between said function, said serial number, and said unique upgrade identifier wherein a valid enabling code is produced only for a compatible combination of said software upgrade and said reprogrammable device.

7. A method for locally upgrading software in an implanted reprogrammable device (IRD) having a serial number, comprising the steps of:
distributing removable media containing an upgrade software program for said IRD and having a unique upgrade identifier;
receiving said serial number and said unique upgrade identifier; and
communicating an enabling code for said upgrade software program used with said IRD, wherein said enabling code comprises a calculated function of said received serial number and said received unique media identifier.

8. The method of claim 1, further comprising the steps of:
locally storing a plurality of said removable media containing a plurality of said upgrade software programs;
downloading information from said IRD, said downloaded information comprising a said serial number;
selecting an appropriate software upgrade from said locally stored plurality of said upgrade software programs;
recording said serial number and said unique media identifier;
calculating a first enabling code as a function of said serial number and said unique media identifier;
communicating said serial number and said unique media identifier;
receiving a second enabling code; and
uploading said upgrade software program to said IRD if said first and second enabling codes are correct enabling codes.

9. The method of claim 7, wherein said IRD comprises an implanted medical device.

10. The method of claim 9, wherein said IRD comprises a pacemaker.

11. The method of claim 7, wherein the step of downloading information from said IRD further comprises downloading a history of IRD activity used for selecting an upgrade software program.

12. The method of claim 7, wherein said uploading and said downloading are performed using a programming device.

13. The method of claim 12, farther comprising the step f calculating said function of said unique identifier and said unique media identifier in said programming device.

14. An apparatus for non-invasively changing a heart-stimulating waveform of an implanted reprogrammable medical device (IRMD) having a unique serial number, the apparatus comprising:
an IRMD software upgrade program in machine-readable media having a unique media identifier; and
a programming device operable to calculate a first enabling code from said unique serial number and said unique media identifier and further operable to upload said IRMD software upgrade program into said IRMD.

15. The apparatus of claim 14, further comprising:
a registry;
a first communication link between said programming device and said IRMD; and
a second communication link between said programming device and said registry.

16. The apparatus of claim 14, wherein said IRMD further comprises a waveform generator responsive to an IRMD computer software program to generate said heart-stimulating waveform.

17. The apparatus of claim 14, wherein said programming device further comprises a software installation switch responsive to an enabling signal to enable uploading of said program to said IRMD.

18. The apparatus of claim 15, wherein said programming device is configured to receive a second enabling code from said registry, said programming device further operable to compare said first enabling code with said second enabling code.

19. The apparatus of claim 18, wherein said programming device is further configured to generate said enabling signal if said first enabling code and said second enabling signal match.
